(19) United States
(12) Patent Application Publication
(10) Pub. No.: US 2023/0192763 A1
(43) Pub. Date: Jun. 22, 2023

(54) METHOD FOR OBTAINING PROTEINS FROM A NATURAL MIXTURE OF SUBSTANCES FROM SOY OR FROM SOY MILK

(71) Applicant: GEA MECHANICAL EQUIPMENT GMBH, Oelde (DE)

(72) Inventors: Detlef ULLMANN, Oelde (DE); Ines SPEISER, Verl (DE); Sabine KROHN, Oelde (DE); Stefan PECORONI, Oelde (DE); Steffen HRUSCHKA, Oelde (DE)

(21) Appl. No.: 17/926,175

(22) PCT Filed: May 19, 2021
(86) PCT No.: PCT/EP2021/063341

§ 371 Date: Nov. 18, 2022

(30) Foreign Application Priority Data
May 20, 2020 (DE) 10 2020 113 747.8

(51) Int. Cl. C07K 1/14; C07K 1/36; C07K 14/415; A23J 1/14

(52) U.S. Cl. CPC C07K 1/145; C07K 1/36; C07K 14/415; A23J 1/142

(57) ABSTRACT

Proteins are obtained from soy milk by concentrating the soy milk while increasing the dry matter content. A water-soluble organic solvent is added to the soy milk to form an organic-aqueous suspension in such a way that a shift in the solubility equilibrium occurs due to the addition of the organic solvent and a displacement extraction takes place. The volume of added organic solvent in step iii is selected such that the content of organic solvent of the organic-aqueous suspension is at least 15% by volume. The suspension is adjusted to a pH value of less than pH=7, forming at least one protein phase. The protein phase, with a residual oil content of less than 5 wt. %, based on the dry substance content of the protein phase, is separated from the suspension.

### BACKGROUND AND SUMMARY OF THE INVENTION

Exemplary embodiments of the present invention relate to a method for obtaining proteins from a native mixture of substances from soybeans or from soy milk. The soy milk, in contrast to the commercially available product, is not a composition extensively diluted with water, but a viscous product that is separated directly with a centrifuge, in particular from soybeans. This soy milk is often referred to in the technical literature as “soy base” or “soybean juice”. This soy milk can also be an intermediate product in the method with the native mixture of substances from soy.

It is known to further process the formed okara obtained from soybean plant components, e.g., from soybeans (hulled or unhulled) or from soybean flakes. A protein phase can be obtained from the separated soy milk.

So far known separation methods of proteins from soy milk allow a recovery of about 80 wt. % of the proteins contained in the soy milk. However, oil and oil-accompanying substances such as lipoxygenases are transferred into the protein. The aforementioned enzyme reacts with the oil present and forms an adverse taste in the protein phase. In addition to lipogenases, soy milk may also contain trypsin inhibitor as an ingredient, which interferes with the absorption of proteins in the body. Furthermore, a minimum oil content is required to protect against rancidity in the final product.

WO 2007/113 176 A2 discloses the processing of soybeans to produce soy milk and native proteins. A reduction of the oil content was not carried out in this method.

EP 1 905 312 A1 also proposes a separation of soybeans into soy milk and a protein fraction. An ether extraction instead of hexane was used to remove oil from the protein.

EP 2 717 711 B1 assumes in para. 0010 an aqueous protein dispersion, which is treated with an incomplete displacement extraction in an alkaline milieu with the addition of an alcoholic solvent, in order to bring the oil accompanying substances such as lecithin into solution. This allows the separation of dissolved or dispersed proteins from the hull components.

Finally, DE 10 2013 114 698 A1 discloses a method for obtaining protein from legumes, such as soybeans. In this process, whole beans are comminuted and processed to a slurry. This is then finally subjected to separation to remove the hulls after a pH shift to pH>9 using alcohol. Finally, at acidic pH, precipitation and separation of several fractions including a protein and an oil fraction is carried out.

Exemplary embodiments of the present invention reduce the oil content in the protein phase obtained from soy components in such a way that no undesirable changes in taste occur.

The method according to the invention relates to the recovery of proteins from soy milk, comprising
i. providing soy milk;
ii. adding an aqueous alcohol with a concentration of less than 80% by volume to the soy milk with formation of an organic-aqueous suspension in such a way that a shift of the solubility equilibrium takes place due to the addition of aqueous alcohol and a displacement extraction takes place, wherein the volume of the aqueous alcohol is selected in such a way that the content of the organic solvent of the organic-aqueous suspension after step ii. is at least 15% by volume and that the dry substance content of the suspension is at least 5%, preferably at least 9%;
iii. separating an oil phase or oil from the suspension;
iv. coagulating the proteins to form at least one protein phase by adjusting the suspension to a pH of less than pH=7 or by adding a protein-coagulate-forming salt; and
v. separating the protein phase with a residual oil content of less than 5 wt. %, in particular with a residual oil content of less than 3%, more preferably with a residual oil content of less than 1%, based on the dry substance content of the protein phase from the suspension.

The protein phase exhibits minor flavor changes and a small amount of residual oil.

The provision of soy milk in step i. can be carried out by obtaining soy milk from a native mixture of substances from soy, wherein the native mixture of substances is first comminuted and processed by pH adjustment and addition of a polar solvent, in particular water, to form a flowable alkaline slurry which, in addition to lipids, also contains proteins, lecithin and solids, wherein the processing of the slurry is carried out with the formation of two separate fractions in the form of okara and of soy milk.

The slurry contains all the essential ingredients of the soybean, including oil. When the soybean is hulled, the slurry contains less fiber. Okara is the “solid cake” with insoluble components and the soy milk has a lot of protein with the dissolved and dispersed substances.

In addition, providing the soy milk may comprise concentrating, preferably thickening, soy milk while increasing the dry substance content so that the provided soy milk may have a dry substance content of preferably at least 12 wt. %, preferably at least 15 wt. %. This corresponds to a comparatively relatively viscous soy milk.

Concentration to a dry substance content of at least 15% has been shown to be a particularly efficient way to release the oil components from the protein structure under the prescribed conditions of pH and temperature and alcohol concentration. Proteins are known to comprise a primary, secondary, tertiary, and quaternary structure, which typically retain oil components. By opening the structure under certain conditions, a particularly efficient separation of oil is possible.

The protein structure is further optimized by the concentration, and then the oil separation can be implemented even more successfully by displacement extraction with EtOH. The addition of ethanol natively reduces the dry substance content again, preferably to a dry substance content of at least 8%.

During concentration, the inner surface of the oil droplets may also change, thus enabling better agglomeration. If the oil droplets are sufficiently large, they can agglomerate particularly well into the continuous phase in the centrifugal field and be separated as such.

Particularly preferably, the concentration can be carried out in such a way that the amount of water removed corresponds approximately to the amount of added aqueous alcohol. Thus, despite the addition of a larger quantity of diluted alcohol to achieve an alcohol content of at least 15% by volume, the dry substance value is at least 8%.

The recommended concentration of alcohol to be added is less than or equal to 80% by volume. Higher concentrated alcohols denature the proteins. Alcohols below 50% by volume, on the other hand, give poor results in displacement extraction due to the high dilution factor.

In the following, the further processing of the soy milk is explained.

According to the invention, the organic solvent is added as a diluted alcohol, preferably with an alcohol content in vol. % of at least 30%, preferably between 50-80%.

At 50-80% by volume, recovery of the alcohol can advantageously be carried out under vacuum.

Preferably, the organic water-soluble solvent is an aliphatic alcohol, in particular with a chain length of less than six carbon atoms, and/or isopropanol.

The pH adjustment in step iv. can preferably be made to a pH value of more than pH=3.5, preferably between pH=3.8 to 6.0, in particular between pH=4.2 and 4.7.

Concentration may be accomplished by reducing the water content of the soy milk, reducing the weight by at least 20 wt. %, preferably at least 40 wt. %, to a dry substance content of at least 12 wt. %, more preferably at least 15 wt. %.

The content of the organic water-soluble solvent in the suspension after step iii. may be more than 15% by volume, preferably between 25-45% by volume. This allows a clear oil phase to be separated. At lower concentrations, separation of an oily phase in the form of a cream occurs, but this is associated with a product loss of proteins.

Insofar as soy milk is prepared in step i. from a flowable okara-containing slurry, it is advantageous if the flowable alkaline slurry in step i. or the soy milk prepared therefrom has a pH value of more than pH=8, in particular between pH=8.2 to 9.8. However, in a further variant of the invention, it is also possible to assume an acidic soy milk without prior pH adjustment with a pH value around 6.7. For this soy milk, too, it is recommended to adjust the pH value for precipitation after ethanol has been added, in particular to the preferred range of pH=3.8 to 6.0.

After the addition of the water-soluble organic solvent in step iii., in particular immediately after the addition of the water-soluble organic solvent, a sequence may comprise at least one malaxation, preferably with a stirring speed of less than 100 rpm. The sequence may particularly preferably further comprise at least one intensive stirring, preferably at a stirring speed greater than 500 rpm. The chronology within the sequence can also be reversed, i.e., first intensive stirring and then malaxation, or a multiple sequence of intensive stirring followed by a single malaxation, or a sequence of malaxation followed by a single intensive stirring. This sequence achieves a particularly complete separation of oil, in that oil droplets are better separated from the protein molecules and then agglomerate by slow stirring to form larger oil droplets and finally an oil phase.

Regardless of the repetitions of the sequences or whether this sequence takes place at all, malaxation should always take place and this should preferably end the sequence.

Malaxation can preferably be carried out at a stirring speed of less than 50 rpm, preferably between 10-30 rpm. Malaxation can also be carried out at 20-70° C., especially preferably at 40-70° C., for particularly optimum agglomeration of oil.

Preferably, malaxation can be performed in a time interval between 5-30 min. Longer malaxation has no further beneficial effect on the formation of the oil phase.

Intensive stirring, on the other hand, can be performed at stirring speeds greater than 1000 rpm, in particular between 1000-12000 rpm.

The aforementioned sequence can be repeated several times, in particular at least 3 times, to achieve additional optimization of oil separation from the suspension. However, the aforementioned sequence should ideally include at least one malaxation.

Furthermore, before step iv. and preferably after the sequence of at least one malaxation and at least one intensive stirring, an oil phase can be separated from the suspension, in particular centrifugally. For reasons of the high viscosity of the suspension, a decanter, in particular in the form of a solid bowl centrifuge, or a separator is advantageously suitable for this purpose.

Centrifugal separation can be carried out particularly optimally at a temperature of more than 20° C., preferably between 35-80° C., especially preferably between 35-45° C. Especially in the aforementioned ranges, an optimum of processing time and technical effort (ex-operation) has been found.

Before step iv. and preferably after centrifugal separation of the oil phase, dealcoholization can be carried out, preferably by evaporation of the alcohol. This may involve recovery of the organic solvent previously added in the form of alcohol.

After pH adjustment in step iv., the suspension can be transferred to a vessel for precipitation of the protein.

Precipitation can be carried out at a temperature higher than 20° C., preferably between 50-80° C., more preferably between 60-75° C., and also preferably in a period of time between 5-20 min.

The separation in step v. may be a centrifugal separation and preferably performed by a decanter, in particular by a solid bowl screw centrifuge.

Centrifugal separation can be performed in a centrifuge or decanter with a separation range, the so-called cut-off value, between 1.0 to 100 μm.

Following protein separation, drying can take place.

The protein phase separated in step v. may have less than 0.5 wt. % lecithin.

Steps i. to iii., and preferably also the provision of the alkaline slurry, can be carried out in a temperature range between 35° C. to a maximum of 85° C.

The polar solvent for the formation of the flowable slurry before step i. can preferably be water, wherein the volume of aqueous polar solvent is selected in such a way that a dilution factor of more than 0.2, preferably at least 2.0, is obtained, based on the concentrated slurry. The crude slurry usually has a dry substance content of about 8-9% dry substance, then it is concentrated and diluted again with polar solvent. Then it should again have dry substance content of not less than 5%, preferably between 7% to 13%, more preferably at least 8%, especially 9% (+/−1). However, a higher dry substance content is not disadvantageous.

For a dry substance content of more than 5%, it is particularly recommended to work at temperatures around 70° C., while for a dry substance content of at least 8%, it is also possible to work at temperatures below 70° C., which is advantageous from a process engineering point of view.

The native substance mixture can be comminuted by wet grinding, which is gentle on the product.

The separation in step v. can be started within 60 min, especially within 30 min, after performing step iv. to avoid side reactions.

In particular, the protein phase is obtained without the addition of hexane, so that there are no further health concerns when the end product is used as feed or food.

The extraction and processing of oils and fats according to the guidelines of organic farming allows in particular mechanical production steps. With a few exceptions, the use of chemical additives is prohibited. These include extraction solvents such as hexane and ether.

Adjusting the pH in step iv. can be carried out by adding an organic or inorganic acid, for example a fruit acid.

A preferred embodiment variant of the method has the following sequence of steps:
i. Providing soy milk, preferably with a pH greater than pH=7.2;
ii Concentrating soy milk while increasing the dry substance content;
iii. Adding short-chain alcohol having 1-5 carbon atoms with formation of an aqueous-alcoholic suspension, in particular with addition of ethanol and/or isopropanol, wherein the alcohol content of the alcoholic-aqueous suspension after step iii. is at least 15% by volume, preferably at least 20% by volume,
iii.i Sequence consisting of at least one malaxation with a stirring speed of less than 100 rpm.
iii.ii Separating, in particular first centrifugal separating, of oil from the suspension to form a substantially oil-free suspension having an oil content of less than 5 wt. %, preferably less than 3 wt. % in dry matter;
iii.iii Optional reduction of alcohol content, preferably by distillation, more preferably with recovery of alcohol;
iv. Adjusting the suspension to a pH value of less than pH=7, preferably to a pH value between pH=3.5 to pH=6; by forming at least one protein phase and at least one liquid phase;
iv.i Optional precipitation of the protein phase as a solid phase;
v. Separating, in particular second centrifugal separating, of a protein phase with a residual oil content of less than 5 wt. % from the suspension, preferably between 1-3 wt. %, based on the dry substance of the protein phase, as well as
vi. Drying of the protein phase to a protein

Further according to the invention is a method for obtaining proteins from a native mixture of substances from soy, wherein the native mixture of substances is first comminuted and processed by adjusting the pH to the basic range and adding a polar solvent to form a flowable alkaline slurry which, in addition to lipids, also contains proteins, lecithin and solids, wherein the method comprises the following further steps:
A Processing the slurry with the formation of two separate fractions in the form of okara and soy milk, and
B Processing the soy milk into a protein phase according to the aforementioned methods of the invention.

Further advantageous designs of the above method are described below. The pH adjustment for producing the flowable alkaline slurry can preferably be carried out by adding an alkali, such as NaOH or an NaHCO₃ solution.

In addition to lipids, this slurry also contains proteins, lecithin, and solids.

During the separation into soy milk and okara, a large part of the proteins is separated from the slurry with the soy milk dissolved and/or dispersed. With an additional washing process of the okara, the protein yield in the soy milk can be increased to well over 70%.

Some of the proteins remain in the okara and can be extracted separately from the further processing of the soy milk.

Steps i) to iii), and preferably also the preparation of the alkaline slurry, can preferably be carried out in a temperature range of up to 85° C. In particular, at least the preparation of the alkaline slurry can be carried out at less than 15° C. in order not to adversely affect the taste of the final protein product or other by-products, such as the okara.

Typically, soy milk is boiled or heated at the time of provision to neutralize the trypsin inhibitors, as these would otherwise interfere with digestion when the protein is consumed. This can optionally also be carried out during provisioning in step i).

The polar solvent for forming the flowable slurry is preferably water, with the volume of water to bean being selected such that a dilution factor of more than 2.0, preferably at least 3.0, in particular between 3.5 to 7.5, is obtained. Preferably, the polar solvent for forming the slurry can be recovered in the process, e.g., by an evaporator. For example, after separation of the protein phase in step v., it is possible to recover both the polar solvent for forming the flowable slurry, and the alcohol used, by fractional distillation and feed them to the various stages of the process. This also reduces disposal costs, among other things.

The native mixture of substances can be comminuted advantageously and in a way that is gentle on the product by wet grinding.

The present method has the particular advantage that the addition of hexane and ether can be completely dispensed with for obtaining the protein phase. This means that no cost- and labor-intensive additional steps have to be carried out to remove this substance from the product again. Hexane is harmful to health and consequently undesirable in products in the feed and food industry.

Further according to the invention is a method for obtaining proteins from soy milk with a residual oil content of more than 3% in dry substance. Soy milk typically always contains a residual oil content far beyond 3% in dry substance mostly around 20% oil/DS (dry substance). Ideally, soy milk can be obtained by the above method, as this soy milk is particularly rich in dry substance and protein. However, a soy milk can also be processed with the method according to the invention that has not been obtained from a flowable alkaline-aqueous slurry and has been separated according to step i. of the preceding-described method.

It is understood that all variants, in particular the variants of the aforementioned steps described as advantageous, which can be applied in the aforementioned method for obtaining proteins from a native mixture of substances from soy, can also be applied advantageously in the aforementioned method for obtaining proteins from soy milk.

Also, according to the invention is a soy protein powder with an oil content of max. 5% oil in the dry matter and a protein content of at least 70% protein in the dry matter, wherein the soy protein powder is absolutely hexane-free. Since no hexane is used in the aforementioned method, no hexane (even in the ppm range) is present in the product. No aforementioned product with this oil content is known to date.

### BRIEF DESCRIPTION OF THE DRAWING FIGURES

Further advantages, features and details of the invention will be apparent from the following description, in which several exemplary embodiments of the invention are explained in more detail with reference to the accompanying drawing. The person skilled in the art will expediently also consider the features disclosed in combination in the drawing, the description and the claims individually and combine them to form useful further combinations. The drawings show as follows:

FIG. 1 shows a process diagram of various preparation steps for providing soy milk during the production of protein from soy; and

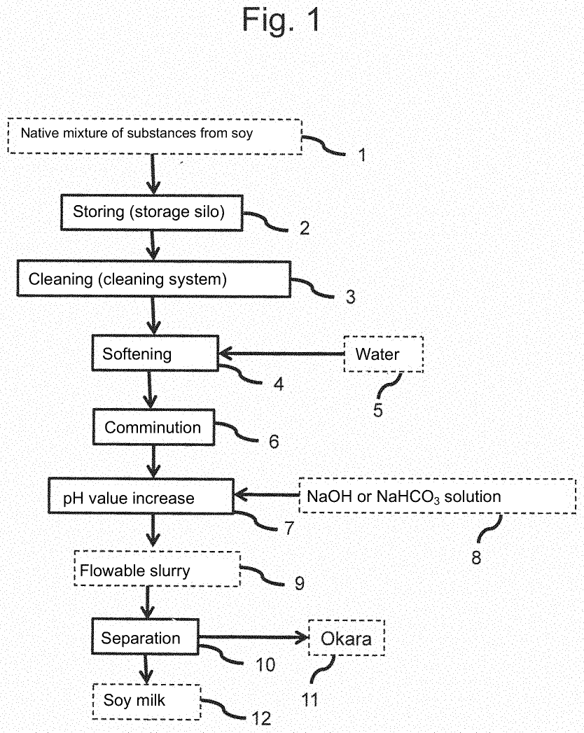

FIG. 2 shows a process diagram for the production of a protein powder from soy milk as a variant of the method according to the invention.

### DETAILED DESCRIPTION

Proteins usually have a primary, secondary, tertiary, and possibly also a quaternary structure. The structural composition depends on the ingredients, such as the oil content, the type of oil (polarity), the type and concentration of the other ingredients in the suspension, the dilution factor, the polarity of the solvent, the pH value, the temperature, and many other factors. The structural composition determines the extent to which individual ingredients such as oil and other non-polar as well as low-polarity substances can be released. If the protein is present in a more compact structure, it is more difficult to release an oil from the protein structure. Therefore, the conditions of protein production can vary greatly depending on the plant variety.

The following method describes an optimized extraction of proteins from soy, in particular soybeans. Alternatively, the method can also start with soy milk as an intermediate product.

In an optional first step, a native mixture of substances from soy 1 is provided. In the present invention, a native mixture of substances from soy is understood to mean in particular soybeans in the hulled or unhulled state, but also so-called soybean flakes. A mixture of substances is referred to when granular components are present, e.g., as a heap or as bulk material.

For example, such a mixture of substances from soy 1 may consist of 38 wt % protein, 18 wt % fat, 15 wt % insoluble hydrocarbons, 15 wt % soluble hydrocarbons, and 14 wt % other ingredients

The soy mixture can first be stored in a storage silo 2 and then passed through a cleaning system 3, which removes dirt from the soybeans or flakes and separates stones and trimmings. This can be carried out, for example, by washing and screening the mixture of substances.

In an optional second step, the native mixture of substances can be softened 4. For this purpose, water 5, preferably water at a temperature of less than 20° C., preferably less than 15° C., can be added to the mixture of substances. This reduces the enzyme activity, in particular the lipoxygenase activity.

Softening 4 is preferably carried out within a period of at least 3 h, preferably from 4 to 10 hours. The use of dehulled soybeans or soy flakes is preferably recommended for softening the mixture. These require only about 3.5 to 4.5 h for softening. Unhulled soybeans require longer to soften. Softening of soybeans allows better conditions during subsequent grinding. The grinding of softened plant components is generally referred to as wet grinding.

As the amount of water 5 used, a weight ratio of at least 2:1 (water to soy) or more, e.g., 3:1, is recommended in relation to the amount of soy.

In a third step, comminution 6 of the components of the soy of the mixture of substances 1 takes place. This can preferably be carried out as wet grinding in the form of cold grinding, warm grinding, or hot grinding.

Cold grinding is preferably carried out at temperatures of less than 15° C. This reduces the enzyme activity, but leads to a reduction in yield.

Warm grinding is preferably carried out at temperatures between 30-50° C., preferably at about 40° C. As a result, due to the increased enzyme activity, a change in flavor occurs as a bean-like taste in the subsequently produced soy milk. This may be desirable to some consumer groups. At the same time, the higher temperature allows yield improvement.

Hot grinding takes place at temperatures of over 80° C. This deactivates enzymes. In this variant, hot water, preferably at more than 95° C., is added to the soy components and preferably held for several minutes, e.g., at least 4 minutes. This variant also reduces the yield compared to warm grinding.

The comminution 6 by grinding can optionally and preferably take place in at least two stages. The first stage can be a pre-grinding in a disk mill.

If a predetermined average particle size is reached, further fine grinding can be carried out in a colloid mill. In this process, cells are opened, thereby increasing the yield.

Before, during or after comminution, the pH 7 of the batch or the comminuted batch can be increased in a fourth step. In this process, the pH value is increased for reasons of yield optimization. The higher pH value enables, among other things, improved solubility of the proteins in water. To increase the pH value, it is preferable to add an alkali, e.g., NaOH solution, or a buffer solution 8, for example a sodium hydrogen carbonate solution.

The result of the aforementioned steps is a flowable alkaline slurry 9, which in a fifth step is fractionated at least into okara 11 and soy milk 12. Further advantageous for the yield of oil-free protein from soy milk is the proportion of water addition 5 in the preparation of the flowable slurry 9. Thus, experimental results have shown that when changing from a dilution factor of 2 to a dilution factor of 3 in the addition of water prior to fractionation of the slurry into okara and soy milk, up to 10% more protein can be obtained as oil-free product from the soy milk. The 10% more proteins refer to the total weight of proteins in the slurry mixture as the starting material. A further increase in the amount of water brings further increases in yield, but from a water ratio greater than 7 to 1 the process becomes unfavorable from a process engineering point of view.

At least one first decanter, preferably a solid bowl screw centrifuge, is used for separation 10 into the two aforementioned fractions. The first decanter preferably has a horizontal position or an axis of rotation inclined to the horizontal position by up to 25°. The feed of the slurry occurs axially and the discharge of the solids, i.e., the okara, as well as the discharge of the soy milk occurs radially. The discharge of the solids can take place in a region of a first end of the decanter and the discharge of the soy milk in a region of a second end of the decanter.

In addition to the further treatment of soy milk 12 according to the invention, okara 11 can also be further processed in a single-stage or multi-stage process. In the single-stage process, the okara 11 is separated from the soy milk in the decanter as described above and discharged from the decanter. Here, a screw pump can be used for removal. A protein yield for the method of over 70% can be achieved. Alternatively, it is also possible to slurry the separated okara 11 in water again and to post-treat it with a second decanter. The separated soy water can be returned to the method for softening. The protein yield with this variant of the method can be up to approx. 80%.

The slurry 9 supplied to the feed of the first decanter may have a content of insoluble components of 25-30% by volume. The temperature of the supplied slurry can preferably be more than 75° C.

The dry substance content of the soy milk 12 at the outlet of the first decanter is preferably >5% m/m, preferably at least 8%, ideally at 8 to 10.5% m/m.

Optionally, in an optional sixth step not shown in detail, the soy milk may be treated by ultra-high temperature heating and/or deodorization. In this process, hot steam may be introduced into the soy milk to deactivate enzyme activity. The product can be heated to more than 100° C., preferably between 120-140° C. In this way, deactivation of the trypsin inhibitor can be achieved. Preference is given to direct heating by steam introduction as described. The temperature is maintained for less than 60 seconds, preferably less than 10 seconds. Thereafter, the soy milk can be cooled again. This can preferably be carried out by a vacuum cooling system for direct cooling without additional refrigerants in the form of flash cooling.

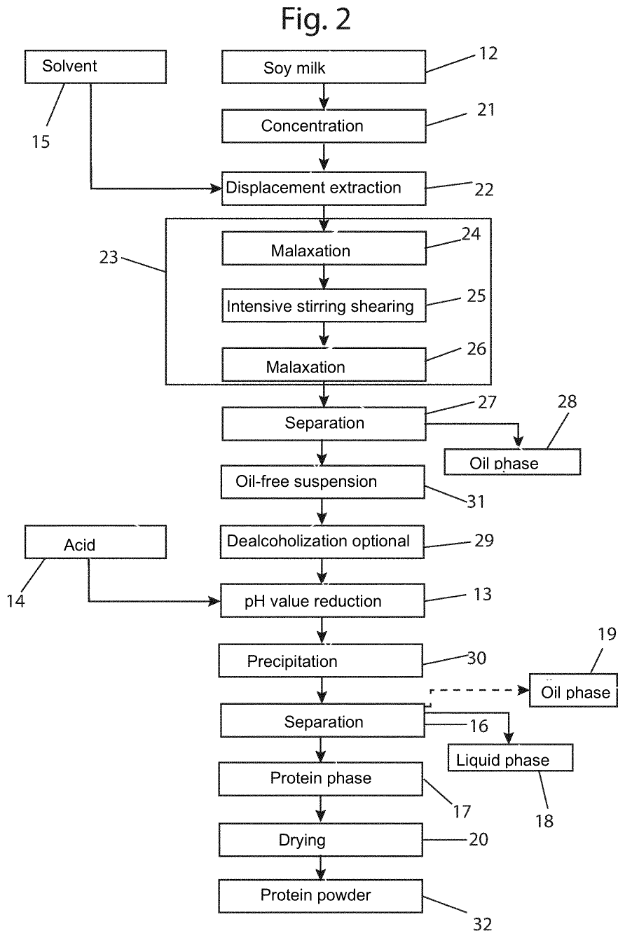

An embodiment variant according to the invention for processing soy milk is shown in FIG. 2.

In a seventh step (step ii), the amount of soy milk is then concentrated down 21, e.g., by evaporating a larger proportion of water from the soy milk 12, so that the weight of the soy milk is reduced by at least 10 wt. %, preferably by at least 40 wt. %. However, there is also soy milk that is already present with 12 or 12.5% DS as raw material. This does not need to be concentrated as much. Overall, a product with a DS content of at least 12%, preferably 15%, is recommended before ethanol extraction.

This step is preferable for the subsequent phase inversion. Surprisingly, it has been shown that a concentration of the amount of soy milk, in particular to a dry substance content of 15 wt. %, is particularly advantageous in order to optimize the method from a process engineering point of view only and to release the oil in an optimum manner.

Following the concentration 21 in the seventh step, an organic solvent 15 is added in an eighth step in the course of a displacement extraction 22. An alcohol with 1-5 carbon atoms is particularly recommended as organic solvent 15, but ethanol and/or isopropanol are especially preferred. It has been shown that when a diluted alcoholic solvent is added, better separation of proteins with low oil content is possible than is the case, for example, with concentrated alcohol. The addition of the solvent in diluted form can therefore preferably be carried out with a solvent content in vol. % of more than 30%, preferably between 50-96%, particularly preferably between 55 to 80%. For example, an ideal alcohol dilution of 60% by volume (+/−5%) has been found for the use of ethanol. During the addition of the solvent, mixing of the suspension can take place to achieve rapid dispersion and homogeneous distribution in the suspension.

The volume and concentration of the solvent, in particular of the alcohol, shall be such that the content of the organic water-soluble solvent 15 in the suspension after step iii. is more than 15% by volume, preferably between 25-45% by volume.

For example, an ideal alcohol concentration in soy milk of 25% by volume (+/−5%) has been found for the use of ethanol.

For comparison purposes, a purely aqueous treatment in the acidic pH range (pH=5) was carried out with the same soy milk 12 without the addition of ethanol. This resulted in a protein product with an oil content of 5.1 wt. % after separation of the solid phase.

After addition of the organic water-soluble solvent in the eighth step, a sequence 22 of malaxation 24 and intensive stirring 25 is carried out in a ninth step. Intensive stirring 25 may in particular comprise intensive mixing. The sequence can be chosen arbitrarily. In particular, intensive stirring can also be carried out first followed by malaxation. It is advantageous to carry out at least one malaxation step 26 immediately before separation 27. However, before this malaxation step 26, as described above, a sequence of a further malaxation 24 and intensive stirring 25 can also be carried out in any order in multiple repetition.

Intensive stirring 25 as defined in the present invention occurs at a speed greater than 500 rpm, more preferably 1000-500 rpm.

In contrast, malaxation 24, 26 according to the definition of the present invention occurs at a stirring speed of less than 100 rpm, preferably less than 50 rpm, more preferably between 10-30 rpm.

During intensive stirring, shear forces occur due to the increased stirring speed, which enable improved separation of the oil from protein structures. In this process, micro-oil droplets are virtually squeezed out of the protein-containing material.

In contrast, due to the low stirring speed, malaxation allows agglomeration of the micro-oil droplets in the suspension into a larger oil droplet up to an oil layer.

In FIG. 2, a sequence of malaxation 24, intensive stirring 25 followed by malaxation 26 is shown once. This sequence of steps 24 and 25 is referred to as step 23 in FIG. 2 and can also be executed 0 to n times. Here, n represents the number of executions in any order.

The temperature during malaxation 24, 26 is preferably 40-70° C. and the time interval of malaxation 24, 26 is also preferably between 5-30 min.

In a tenth step, an oil phase 28 is subjected to separation 27 to form an essentially oil-free suspension 31. If the suspension after the eighth step contains more than 20% by volume of the solvent, in particular of the alcohol, a comparatively clear oil phase 28 can be separated in this step. At concentrations of solvent between 15-20% by volume, the separated oil phase 28 has the consistency of a cream.

The separation can preferably be carried out at a temperature of more than 40° C., preferably between 50-80° C., particularly preferably between 60-75° C., for example at 70° C. This additionally facilitates the separation of oil.

Optionally, a dealcoholization 29 may be performed in an eleventh step. This dealcoholization can be carried out by evaporating or distilling alcohol. In particular, it is possible to at least partially recover the alcohol used in the eighth step. However, the dealcoholization is not absolutely necessary. The protein yield is comparable without this step.

Then a final pH adjustment of the basic suspension is carried out in a twelfth step 13, if this has not already been carried out before step 10. The adjustment of the pH value should enable a shift into the acidic range, i.e., below pH=7, for acid precipitation.

A food-grade acid 14 is preferably used to adjust the pH value. This is preferably a fruit acid and particularly preferably citric acid. However, the use of an inorganic acid, such as HCl, is also possible. The pH value should preferably be at a pH value of more than pH=3.5, preferably between pH=3.8 to 6.0, in particular between pH=4.2 and 4.7, since the configuration of the protein structure in this range allows optimum oil separation and the proteins have a structure which facilitates further processing, e.g., centrifugal separation and drying.

The concentration of the acid is preferably 5-50% by volume.

During acid addition, mixing of the suspension can take place to achieve rapid adjustment of the pH value and homogeneous distribution in the suspension.

Alternatively, a protein coagulant-forming salt, e.g., calcium sulfate, calcium chloride, magnesium chloride and/or calcium glyconate, can be used instead of an acid while adjusting the pH.

The concentration of the added salt relative to the total mass of the soy milk is preferably at least 0.15 mass %, more preferably between 0.2 to 2.5 mass %.

Following the addition of acid or coagulant, precipitation 30 of the proteins can be carried out in a thirteenth step, initially by gravity. For this purpose, the suspension is transferred to a settling tank. Due to the change in pH, the proteins can settle out of the suspension. This process can preferably take 5-20 min. Optimal settling of the now particulate proteins can take place at temperatures above 40° C., preferably between 50-80° C., particularly preferably between 60-75° C.

After precipitation, a separation 16 into a protein fraction 17 and a liquid fraction 18 takes place in a fourteenth step. This can be carried out in a centrifuge, in particular in a separator, or in a filtration plant. Optionally, separation of an oil phase 19 or oil fraction as a further valuable product can also take place at this point, although the oil components can also remain in the liquid phase. This separation of the oil fraction 19 can take place alternatively or additionally to the separation of the oil fraction 28 in the tenth step 27.

Separation 16 can preferably be started within 60 min, preferably within 30 min, after the pH has been adjusted. Precipitation of protein to form a pro-particle phase and separation, e.g., by centrifugation, can also be carried out at the same time, e.g., by introducing acid or coagulating salt into a centrifuge or separator or decanter.

The separated protein phase 17 preferably has a lecithin content of less than 0.5 wt. %.

Then, in a fifteenth step, drying 20 of the protein phase 17 can be carried out. Spray drying or, particularly gentle on the product, grinding drying can be used.

The oil-free protein phase thus obtained can be used as protein powder 32, in particular in the food and feed industry.

In the context of the present invention, oil-free refers to a protein phase with less than 3 wt. %, in particular less than 1 wt. %, of oil in the dried protein phase.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

### LIST OF REFERENCE SIGNS

1 Soy
2 Storage silo
3 Cleaning system
4 Softening
5 Water
6 Comminution
7 pH value increase
8 NaOH or NaHCO₃ solution
9 Alkaline slurry
10 Separation
11 Okara
12 Soy milk
13 pH value reduction
14 Acid
15 Organic solvent
16 Separation
17 Protein phase
18 Liquid phases
19 Oil phase
20 Drying
21 Concentrating the soy milk
22 Displacement extraction
23 Sequence
24 Malaxation
25 Intensive stirring
26 Malaxation
27 Separation
28 Oil phase
29 Dealcoholization
30 Precipitation
31 Oil-free suspension
32 Protein powder

1-35. (canceled)

36. A method for obtaining proteins from soy milk, the method comprising:
i. providing soy milk;
ii. adding an aqueous alcohol with a concentration of less than 80% by volume to the soy milk with formation of an organic-aqueous suspension in such a way that a shift in solubility equilibrium takes place due to the addition of aqueous alcohol and a displacement extraction takes place, wherein a volume of the aqueous alcohol is selected in such a way that content of the organic solvent of the suspension after step ii. is at least 15% by volume and that dry substance content of the suspension is at least 5%;
iii. separating an oil phase from the organic-aqueous suspension;
iv. coagulating the proteins to form at least one protein phase by adjusting the suspension to a pH value of less than pH=7 or by adding a protein-coagulate-forming salt; and
v. separating the at least one protein phase with a residual oil content of less than 5 wt. %, based on the dry substance content of the protein phase, from the suspension.

37. The method of claim 36, wherein the provision of soy milk in step i. is performed by obtaining the soy milk from a native mixture of substances from soy, wherein the native mixture of substances is first comminuted and is processed by pH adjustment to a basic pH value of greater than pH=7.2 and addition of a polar solvent to form a flowable alkaline slurry which, in addition to lipids, also contains the proteins, lecithin, and solids, wherein the processing of the slurry is carried out with the formation of two separate fractions in a form of okara and of soy milk.

38. The method of claim 36, wherein the aqueous alcohol is an aliphatic alcohol with a chain length of less than six carbon atoms or isopropanol.

39. The method of claim 36, wherein the addition of the aqueous alcohol is performed with an alcohol content in percent by volume of 50 to 80%.

40. The method of claim 36, wherein the pH adjustment in step iv. is to a pH value between pH=4.2 and 4.7.

41. The method of claim 36, wherein the provided soy milk is adjusted to a dry substance content of more than 15% by concentrating while reducing water content of the soy milk.

42. The method of claim 36, wherein a content of the aqueous alcohol in the suspension after step ii. is between 25-45% by volume.

43. The method of claim 36, wherein the soy milk provided in step i) has a pH value between pH=8.2 to 9.8.

44. The method of claim 36, wherein immediately after the addition of the aqueous alcohol in step ii., a sequence of at least one malaxation is performed at a stirring speed of less than 100 rpm.

45. The method of claim 44, wherein at least one intensive stirring is performed with a stirring speed of more than 500 rpm.

46. The method of claim 44, wherein the at least one malaxation is performed at a stirring speed between 10-30 rpm.

47. The method of claim 44, wherein the at least one malaxation is performed at 40-70° C.

48. The method of claim 47, wherein the at least one malaxation is performed in a time interval between 5-30 min.

49. The method of claim 45, wherein the intensive stirring is performed at stirring speeds between 1000-12000 rpm.

50. The method of claim 44, the sequence comprises the at least one malaxation and at least one intensive stirring.

51. The method of claim 50, wherein before step iv, and after the sequence of the at least one malaxation and the at least one intensive stirring, the separation of an oil phase from the suspension is performed.

52. The method of claim 51, the separation of the oil phase prior to step iv. is performed by a centrifugal separation in a decanter or a separator.

53. The method of claim 52, wherein the centrifugal separation is performed at a temperature between 35-45° C.

54. The method of claim 52, wherein before step iv and after the centrifugal separation of the oil phase, dealcoholization is performed by evaporation of the alcohol.

55. The method of claim 36, wherein after step iv. the suspension is transferred to a vessel for precipitation of the protein.

56. The method of claim 55, wherein the precipitation is performed at more at a temperature between 60-75° C.

57. The method of claim 56, wherein the precipitation is performed in a period of time between 5-20 min.

58. The method of claim 36, wherein the separation in step v. is a centrifugal separation.

59. The method of claim 58, wherein the centrifugal separation is performed using a centrifuge or decanter with a separation range cut-off value between 1.0 to 100 μm.

60. The method of 36, further comprising:
drying the at least one protein phase after step v.

61. The method of claim 36, wherein the at least one protein phase separated in step v.) has less than 0.5 wt. % lecithin.

62. The method of claim 36, wherein the steps i) to iii) and also a provision of an alkaline slurry in the provided soy milk are performed in a temperature range from 35° C. to a maximum of 85° C.

63. The method of claim 37, wherein the polar solvent prior to step i. for forming the flowable alkaline slurry in the provision of soy milk is water, wherein a volume of water is selected such that a dilution factor of more than at least 3.0 is obtained.

64. The method of claim 36, wherein the separation in step v. is started within 30 min after performing step iv.

65. The method of claim 36, wherein the protein phase is obtained from the soy milk without addition of hexane or ether.

66. The method of claim 36, wherein the adjustment of the pH value in step iv. is performed by adding an organic or inorganic acid.

67. A method for obtaining proteins from soy milk, the method comprising the following sequence of steps:
i. providing of the soy milk, which has more than pH=7.2;
ii. concentrating the soy milk while increasing the dry matter content;
iii. adding an aqueous short-chain alcohol having 1-5 carbon atoms to the concentrated soy milk to form an aqueous-alcoholic suspension by adding ethanol or isopropanol, wherein alcohol content of the alcoholic-aqueous suspension after step iii is at least 20% by volume,
iii.i performing a sequence of at least one malaxation with a stirring speed of less than 100 rpm or at least one intensive stirring with a stirring speed of at least 500 rpm;
iii.ii performing a first centrifugal separation of oil from the aqueous-alcoholic suspension to form a substantially oil-free suspension having an oil content of less than 3 wt. % in dry matter;
iii.iii reducing alcohol content of the substantially oil-free suspension by distillation to recover alcohol;
iv. adjusting the reduced alcohol content, substantially oil-free suspension to a pH value between pH=3.5 to pH=6 by forming at least one protein phase and at least one liquid phase;
iv.i precipitating the protein phase as a solid phase;
v. performing a second centrifugal separating of a protein phase with a residual oil content of less than 1-3 wt. % from the suspension, based on the dry substance of the protein phase; and
vi. drying the protein phase to a protein powder.

68. A method for obtaining proteins from a native mixture of substances from soy, the method comprising:
wherein the native mixture of substances is first comminuted and processed by pH adjustment and addition of a polar solvent to form a flowable alkaline slurry which, in addition to polar lipids, also contains proteins, lecithin and solids,
processing of the flowable alkaline slurry with the formation of two separate fractions in the form of okara and of soy milk; and
processing the soy milk into a protein phase according to the method of claim 36.

69. The method of claim 68, wherein the comminution of the native mixture of substances is performed by wet grinding.

70. A soy protein powder produced by the method according to claim 36, having an oil content of at most 5% oil in the dry matter and a protein content of at least 70% protein in the dry matter, wherein the soy protein powder is absolutely hexane-free and ether-free.
